(19) United States
(12) Patent Application Publication
(10) Pub. No.: US 2024/0003883 A1
(43) Pub. Date: Jan. 4, 2024

(54) CONTROL FOR PROTEIN-BASED ASSAY

(71) Applicant: ZeptoMetrix LLC, Buffalo, NY (US)

(72) Inventors: Kelly A. Cycon, Buffalo, NY (US); Douglas X. MASON, Buffalo, NY (US); Tanya Cruz-Morris, Buffalo, NY (US)

(21) Appl. No.: 18/344,693

(22) Filed: Jun. 29, 2023

Related U.S. Application Data
(60) Provisional application No. 63/490,886, filed on Mar. 17, 2023.
(60) Provisional application No. 63/378,735, filed on Oct. 7, 2022.
(60) Provisional application No. 63/357,415, filed on Jun. 30, 2022.

(51) Int. Cl. G01N 33/569

(52) U.S. Cl. CPC G01N 33/56983; G01N 2469/10; G01N 2496/45

(57) ABSTRACT

A method for producing a positive antigen-based control for detecting a pathogen which includes providing a culture fluid having a pathogen with a first detectable antigen, wherein the culture fluid has a first concentration of the first detectable antigen, and exposing the culture fluid to UV electromagnetic irradiation for a time period sufficient to render the pathogen inactivated, thereby producing a positive antigen-based control, wherein the positive antigen-based control has a second concentration of the first detectable antigen that is no more than about 50% different from the first concentration when determined via ELISA. Also described are positive antigen-based controls and kits having the same.

### CROSS-REFERENCE TO RELATED APPLICATIONS

This application claims priority to U.S. Application No. 63/357,415, filed on Jun. 30, 2022, U.S. Application No. 63/378,735, filed on Oct. 7, 2022, and U.S. Application No. 63/490,886, filed on Mar. 17, 2023, the contents of which are expressly incorporated herein in their entirety.

### BACKGROUND

Due to the rapidly evolving nature of the severe acute respiratory syndrome coronavirus 2 (SARS-CoV-2) pandemic, molecular and rapid assays have recently undergone development at the same velocity and intensity for the first time in history. The development of antigen-based assays in particular has garnered much attention, although suitable positive controls for such assays remain a challenge. For example, common positive controls for antigen-based assays utilize recombinant proteins. However, while such controls may confirm an assay's ability to detect free protein, these controls often fail to provide reliable data on an assay's ability to detect whole organisms, where detection of a selected antigen may be complicated by other components of the organism (e.g., other proteins contained and/or produced by the organism).

On the other hand, the use of whole organisms as positive controls provides its own challenges. For example, many laboratories require non-infectious samples for antigen-based assay development, whereas current methods for rendering pathogens such as SARS-CoV-2 non-infectious have deleterious effects on proteins that would be targeted by the assays. In addition, the quantification of non-infectious organisms is an obstacle, as conventional pathogenic measurements (e.g., TCID₅₀) are often inconsistent with actual pathogen concentration. This inconsistency provides a significant challenge in the production of reliable positive controls.

There is thus a need in the art for safe, reliable positive controls for the development, testing, and use of antigen-based assays.

### SUMMARY

Disclosed herein is a method for producing a positive control for detecting a pathogen. The method utilizes a culture fluid containing a target pathogen, such as an infectious virus, that has at least one detectable antigen, such as a protein, at a first concentration. The method may further include exposing the culture fluid to UV irradiation for a time period sufficient to render the pathogen inactivated, thereby producing a non-infectious positive control. The positive control may contain a second concentration of the detectable antigen.

### BRIEF DESCRIPTION OF THE DRAWINGS

FIG. 1A shows an example first phase of an ELISA procedure according to aspects of the present disclosure.

FIG. 1B shows an example second phase of an ELISA procedure according to aspects of the present disclosure.

FIG. 1C shows an example third phase of an ELISA procedure according to aspects of the present disclosure.

FIG. 1D shows an example fourth phase of an ELISA procedure according to aspects of the present disclosure.

FIG. 2 shows the TCID₅₀ measurements as described in Example II.

FIG. 3 shows the TCID₅₀ measurements as described in Example III.

FIG. 4 shows the SARS-CoV-2 qPCR measurements as described in Example III.

FIG. 5 shows the ELISA measurements as described in Example III.

### DETAILED DESCRIPTION

Disclosed herein is a method for producing a positive control for detecting a pathogen, the pathogen having at least one detectable target antigen, such as a protein. The method includes providing a culture fluid having the pathogen, wherein the culture fluid has a first concentration of the detectable target antigen, and exposing the culture fluid to electromagnetic irradiation for a time period sufficient to render the pathogen inactivated, thereby producing a positive control. The positive control may have an acceptable second concentration of the detectable target antigen. According to some aspects, the first detectable target antigen concentration and the second detectable target antigen concentration are determinable via an immunoassay, such as an enzyme-linked immunosorbent assay (ELISA).

Also disclosed herein are positive controls that include an inactivated pathogen, wherein the positive control is provided with at least two quantification values. The present disclosure is also directed to positive controls provided by the methods described herein and methods of using the positive controls as described herein, such as using the positive controls in a protein biomarker assay.

As used herein, the term “pathogen” refers to any agent that, in its natural state, may cause infection and/or disease. Example pathogens include viruses, bacteria, fungi, protozoa, and yeast. A “virus” according to the present disclosure includes enveloped and nonenveloped viruses and those containing RNA and/or DNA as nuclear material. Example viruses useful according to the present disclosure include, but are not limited to, SARS-CoV-2, human immunodeficiency virus (HIV), Influenza viruses (Flu A and B), Respiratory Syncytial Virus (RSV), cytomegalovirus (CMV), human lymphotrophic virus (HTLV), Epstein-Barr virus (EBV), and herpes viruses (HSVs).

The method of the present disclosure may include providing a culture fluid having a pathogen as described herein. The culture fluid may be a natural media, an artificial media, or a combination thereof. Non-limiting examples of culture fluids according to the present disclosure include Minimum Essential Medium (MEM), Dulbecco's Modified Eagle Medium (DMEM), RPMI medium, IMDM, PBS, DPBS, HBSS, EBSS, and combinations thereof. According to some aspects, the culture fluid may be purified.

In some non-limiting examples, the pathogen may be isolated from an infected biological fluid. Non-limiting examples of biological fluids include blood, serum, plasma, defibrinated plasma, stabilized plasma pool, cerebrospinal fluid, urine, saliva, semen, mucus, and sputum. Additionally, or alternatively, the pathogen may be cultured as known in the art.

As described herein, the pathogen may be provided in a purified culture fluid, wherein the purified culture fluid is purified as known in the art. While not particularly limited, purification may include removing cells and cell debris (e.g., by separation techniques based on size and/or mass, such as filtration and low-speed centrifugation), concentrating the pathogen (e.g., by filtration and/or high-speed centrifugation), and/or performing ultracentrifugation and/or density gradient purification techniques, as known in the art.

The method of the present disclosure may include providing a selected concentration of pathogen in the culture fluid as described herein. According to aspects, the concentration of pathogen contained in and/or provided to the culture fluid may be determined by measuring the Tissue Culture Infectious Dose (TCID₅₀) of the pathogen. In some non-limiting examples, the pathogen may be provided in the culture fluid at a TCID₅₀ of between about 10⁴ and 10⁸ units/mL.

The culture fluid having a pathogen as described herein will include a first concentration of a detectable target antigen. It should be understood that the first concentration as described herein may correspond with the concentration of detectable target antigen prior to inactivation of the pathogen. As used herein, the term “target antigen” refers to any antigen contained by and/or produced by a pathogen that may be targeted by an antigen biomarker assay. A “detectable target antigen” refers to a target antigen that may be identified and/or quantified by an antigen biomarker assay. As used herein, the term “antigen biomarker assay” refers to an assay designed to detect a target antigen, and in particular, an assay designed to detect a target antigen in order to detect infection by a pathogen and/or diagnose a disease associated with a pathogen.

The antigen biomarker assays according to the present disclosure are not particularly limited. For example, the antigen biomarker assays may be qualitative and/or quantitative. In some non-limiting examples, the antigen biomarker assays may be used in a research setting and/or a medical setting. Antigen biomarker assay types may include, but are not limited to Lateral Flow Assays and ELISA assays.

The detectable target antigen according to the present disclosure may be any antigen which may be targeted by an antigen biomarker assay as described herein. For example, the detectable target antigen may include a protein, such as a viral proteins. Example viral proteins include, but are not limited to, a structural protein, a nonstructural protein, a regulatory protein, an accessory protein, or a combination thereof. In one non-limiting example wherein the pathogen is SARS-CoV-2, the detectable target antigen may include the nucleocapsid protein (N Protein) and/or the spike protein (S protein). In some non-limiting examples, the target antigen may include a conserved region of a pathogenic protein, such as a viral protein. As used herein, a “conserved region” of a pathogenic protein refers to sequence of amino acids in a pathogenic protein that does not vary between strains of a pathogen.

The method according to the present disclose may include exposing the culture fluid as described herein to electromagnetic irradiation for a time period sufficient to render the pathogen inactivated, also referred to herein as an inactivated pathogen. The term “inactivated pathogen” as described herein refers to a pathogen that has been modified such that it is incapable of causing infection and/or disease in a human. In some non-limited examples, an inactivated pathogen may include a pathogen that has been modified such that it is incapable of meaningful replication, that is, incapable of replication sufficient to cause infection and/or disease in a human.

According to some aspects, inactivation of a pathogen may be determined using a TCID₅₀ assay or an Infectivity assay. As known in the art, such assays involve subjecting a sample of serially diluted virus culture fluid or inactivated pathogen into cells in a 96-well plate format. In some non-limiting examples, the assay may include one initial dilution followed by additional serial dilutions across the remainder of the 96-well plate. According to the Infectivity assay, inactivation corresponds with no cytopathic effect (CPE) observed in any well across the 96-well plate.

According to some aspects, electromagnetic irradiation may include UV irradiation, gamma irradiation, microwave irradiation, IR irradiation, X-ray irradiation, or a combination thereof.

As described herein, UV irradiation may be supplied to the culture fluid via a UV source, including but not limited to a UV lamp. It should be understood that the level of UV irradiation supplied to the culture fluid may depend on, for example, characteristics of the UV source (e.g., wattage of the UV lamp), distance between the UV source and the culture fluid, UV radiation wavelength, volume of culture fluid exposed to UV irradiation, or a combination thereof.

As described herein, the UV source may include a UV lamp, as known in the art. Some non-limiting examples of UV lamps include a Model G30T8 Ultraviolet UV-C Lamp.

According to some aspects, the distance between the culture fluid and the UV source (e.g., a UV lightbulb) during UV irradiation may be between about 1 and 50 inches, optionally between about 10 and 50 inches, and optionally between about 20 and 40 inches. It should be understood, however, that the disclosure is not necessarily limited in this way. In particular, the distance between the culture fluid and the UV source may vary based on UV source, culture fluid volume, or a combination thereof.

According to some aspects, the culture fluid may be exposed to UVA light, UVB light, UVC light, or a combination thereof. It should be understood that UVA light has a wavelength of between about 315 to 400 nm, UVB light has a wavelength of between about 280 to 315 nm, and UVC light has a wavelength of between about 100 and 280 nm. According to some aspects, the culture fluid may be exposed to UV light having a wavelength of between about 200 and 300 nm, optionally between about 225 and 275 nm, and optionally about 254 nm.

In some non-limiting examples, UV exposure may be provided to the culture fluid at a volume of between about 1 and 500 mL, optionally between about 50 and 250 mL, optionally between about 100 and 200 mL, and optionally about 125 mL.

The method according to the present disclosure may include exposing the culture fluid to electromagnetic irradiation for an irradiation time period sufficient to render the pathogen inactivated, as described herein. It should be understood that the irradiation time period sufficient to render the pathogen inactivated may depend at least in part on the level of electromagnetic irradiation supplied to the culture fluid, as described above.

Non-limiting examples of irradiation time periods useful according to the present disclosure include between about 1 and 20 minutes, optionally between about 1 and 15 minutes, optionally between about 5 and 15 minutes, and optionally about 10 minutes. Other non-limiting examples of irradiation time periods useful according to the present disclosure include about 1 minute, optionally about 2 minutes, optionally about 3 minutes, optionally about 4 minutes, optionally about 5 minutes, optionally about 6 minutes, optionally about 7 minutes, optionally about 8 minutes, optionally about 9 minutes, optionally about 10 minutes, optionally about 11 minutes, optionally about 12 minutes, optionally about 13 minutes, optionally about 14 minutes, and optionally about 15 minutes.

According to some aspects, the culture fluid may be agitated (e.g., swirled and/or shaken) for all or a portion of the irradiation time period. Additionally or alternatively, the culture fluid may remain unagitated for all or a portion of the irradiation time period. Additionally, the culture fluid may or may not be placed on ice or kept cold during the electromagnetic irradiation.

It should be understood that the irradiation time period as described herein may be a continuous time period. Alternatively, the irradiation time period of the present disclosure may include two, three, four, or more portions, wherein each portion is separated by one or more steps of the method as described herein. For example, the method may include exposing the culture fluid to electromagnetic irradiation for a first portion of the irradiation time period, agitating the culture fluid, and exposing the culture fluid to electromagnetic irradiation for a second portion of the irradiation time period. Separate portions may be pooled into one volume to increase the batch size.

The method of the present disclosure beneficially provides a positive control having an acceptable second concentration of the detectable target antigen. It should be understood that the second concentration as described herein may correspond with the concentration of detectable target antigen after inactivation of the pathogen as described herein. As used herein, an “acceptable concentration” refers to a concentration of the detectable target antigen that is detectable by and sufficient for use as a positive control in an antigen biomarker assay as described herein. It should be understood that the positive control may refer to a culture fluid containing an inactivated pathogen as described herein. Alternatively, when the inactivated pathogen has been separated from the culture fluid, the positive control may refer to the inactivated pathogen.

According to some aspects, the acceptable second concentration of the detectable target antigen may be within a certain range of the first concentration of the detectable target antigen as described herein. For example, the second concentration may be no more than about 50% different from the first concentration, optionally no more than about 40% different, optionally no more than about 30% different, optionally no more than about 20% different, and optionally no more than about 10% different. In some non-limiting examples, the second concentration may be no more than about 50% different from the first concentration, optionally no more than about 49% different, optionally no more than about 48% different, optionally no more than about 47% different, optionally no more than about 46% different, optionally no more than about 45% different, optionally no more than about 44% different, optionally no more than about 43% different, optionally no more than about 42% different, optionally no more than about 41% different, optionally no more than about 40% different from the first concentration, optionally no more than about 39% different, optionally no more than about 38% different, optionally no more than about 37% different, optionally no more than about 36% different, optionally no more than about 35% different, optionally no more than about 34% different, optionally no more than about 33% different, optionally no more than about 32% different, optionally no more than about 31% different, optionally no more than about 30% different from the first concentration, optionally no more than about 29% different, optionally no more than about 28% different, optionally no more than about 27% different, optionally no more than about 26% different, optionally no more than about 25% different, optionally no more than about 24% different, optionally no more than about 23% different, optionally no more than about 22% different, optionally no more than about 21% different, optionally no more than about 20% different from the first concentration, optionally no more than about 19% different, optionally no more than about 18% different, optionally no more than about 17% different, optionally no more than about 16% different, optionally no more than about 15% different, optionally no more than about 14% different, optionally no more than about 13% different, optionally no more than about 12% different, optionally no more than about 11% different, optionally no more than about 10% different from the first concentration, optionally no more than about 9% different, optionally no more than about 8% different, optionally no more than about 7% different, optionally no more than about 6% different, optionally no more than about 5% different, optionally no more than about 4% different, optionally no more than about 3% different, optionally no more than about 2% different, and optionally no more than about 1% different. According to some aspects, the first concentration and/or the second concentration may be determinable by ELISA.

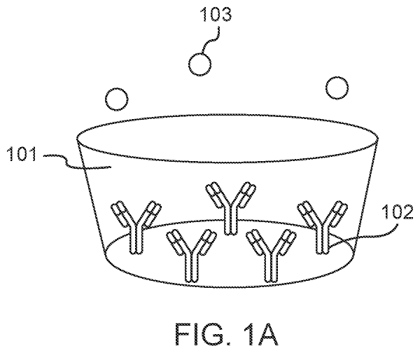

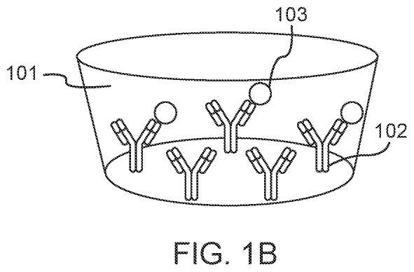

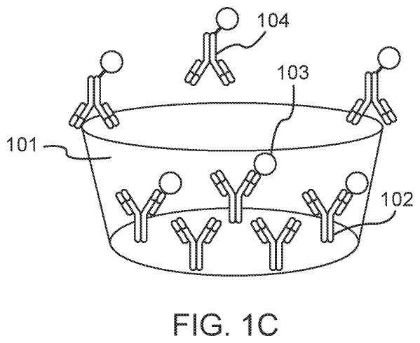

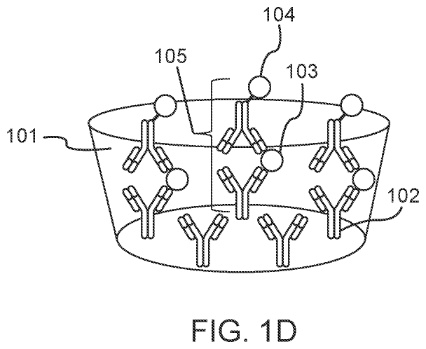

FIGS. 1A-1D show one example schematic of an ELISA protocol useful according to the present disclosure. In this example, a well plate 101 may be pre-coated with an antibody 102 specific for a detectable target antigen, such as a SARS-CoV-2 N protein, thus immobilizing antibody 102 as shown in FIG. 1A. As shown in FIG. 1A, a sample of a positive control containing detectable target antigen 103 may be added to well plate 101, where detectable target antigen 103 may then bind to immobilized antibody 102 as shown in FIG. 1B. As shown in FIG. 1C, after washing well plate 101, a signaling antibody 104 may then be added to well plate 101, such as horseradish peroxidase conjugated anti-SARS-CoV-2 N protein antibody. Well plate 101 may then be washed to remove any unbound signaling antibody 104, leaving behind only antibody-antigen-antibody sandwich complexes 105 as shown in FIG. 1D. It should be understood that each of the sandwich complexes 105 includes an immobilized antibody 102, a detectable target antigen 103, and a signaling antibody 104. Sandwich complexes 105 may be quantified by loading well plate 101 with a substrate solution (e.g., a TMB substrate solution) and developing color. The intensity of color will be proportionate to the number of sandwich complexes 105 immobilized in well plate 101 (and thus, the number of detectable target antigens 103 immobilized in well plate 101).

It should be understood, however, that the present disclosure is not limited to this example. That is, the first concentration of detectable target antigen and/or the second concentration of detectable target antigen may be determined using a tool in addition to or instead of an ELISA. According to some aspects, the first concentration of detectable target antigen and/or the second concentration of detectable target antigen may be determined using any acceptable immunoassay as known in the art. Non-limiting examples of immunoassays according to the present disclosure include a western blot technique, an immunofluorescence assay (IFA), and a lateral flow test (LFT). Additionally or alternatively, the first concentration of detectable target antigen and/or the second concentration of detectable target antigen may be determined by measuring infectivity prior to inactivation, via a polymerase chain reaction (PCR) technique, via chromatography (e.g., high-performance liquid chromatography), via mass spectroscopy, via a total protein assay (e.g., colorimetric or spectrometric), or a combination thereof.

The method of the present disclosure may include one or more steps that provide a second concentration of the detectable target antigen that is closer to the first concentration than would be provided by the same method without such steps. In one non-limiting example, the method may include combining the positive control with an agent that reduces and/or eliminates the breakdown of proteins, including the breakdown of detectable target antigens as described herein. Example agents include, but are not limited to, protease inhibitors. Non-limiting examples of protease inhibitors include aprotinin, bestatin, calpain I, calpain II, chymostatin, E-64, leupeptin, alpha-marcoglobulin, pefabloc SC, pepstatin, TLCK-HCL, trypsin inhibitors, and combinations thereof. In some non-limiting examples, the protease inhibitor includes one or more agents that at least partially inhibit activity of pancreas extract, pronase, thermolysin, chymotrypsin, trypsin, papain, or a combination thereof. In some non-limiting examples, the agent may be provided with the positive control such that the second concentration may be no more than about 50% different from the first concentration, optionally no more than about 40% different, optionally no more than about 30% different, optionally no more than about 20% different, and optionally no more than about 10% different.

According to some aspects, a protease inhibitor may be combined with the culture fluid prior to and/or during electromagnetic irradiation as described herein. Additionally or alternatively, a protease inhibitor may be combined with the positive control.

According to some aspects, the method of the present disclosure may include partially or completely removing the protease inhibitor prior to, during, and/or after electromagnetic irradiation as described herein. Additionally, or alternatively, all or a portion of the protease inhibitor may serve as a component of the positive control as described herein.

The method of the present disclosure may include one or more additional processing steps, including pooling and/or aliquoting samples of the culture fluid as described herein. Additionally or alternatively, the method may include providing the positive control in packaging, such as into sterile vials or tubes. In some non-limiting examples, the method may include separating the inactivated pathogen from the culture fluid and/or providing the positive control as part of a positive control device, such as part of a positive control swab.

The present disclosure is also directed to a positive control obtainable by the method as described herein. The positive control may include a culture fluid as described herein having an inactivated pathogen and a concentration of a detectable target antigen that is within about 50% of a concentration of the detectable target antigen in the same culture fluid prior to the pathogen being inactivated, optionally within about 40%, optionally within about 30%, and optionally within about 20%. Additionally or alternatively, the positive control may include an inactivated pathogen as described herein having concentration of a detectable target antigen that is within about 50% of a concentration of the detectable target antigen in the same pathogen prior to being inactivated, optionally within about 40%, optionally within about 30%, optionally within about 20%, and optionally within about 10%. In some non-limiting examples, the positive control may include an inactivated pathogen as described herein having concentration of a detectable target antigen that is within about 50% of a concentration of the detectable target antigen in the same pathogen prior to being inactivated, optionally within about 49%, optionally within about 48%, optionally within about 47%, optionally within about 46%, optionally within about 45%, optionally within about 44%, optionally within about 43%, optionally within about 42%, optionally within about 41%, optionally within about 40% different from the first concentration, optionally within about 39%, optionally within about 38%, optionally within about 37%, optionally within about 36%, optionally within about 35%, optionally within about 34%, optionally within about 33%, optionally within about 32%, optionally within about 31%, optionally within about 30% different from the first concentration, optionally within about 29%, optionally within about 28%, optionally within about 27%, optionally within about 26%, optionally within about 25%, optionally within about 24%, optionally within about 23%, optionally within about 22%, optionally within about 21%, optionally within about 20% different from the first concentration, optionally within about 19%, optionally within about 18%, optionally within about 17%, optionally within about 16%, optionally within about 15%, optionally within about 14%, optionally within about 13%, optionally within about 12%, optionally within about 11%, optionally within about 10% different from the first concentration, optionally within about 9%, optionally within about 8%, optionally within about 7%, optionally within about 6%, optionally within about 5%, optionally within about 4%, optionally within about 3%, optionally within about 2%, and optionally within about 1%. In some non-limiting examples, the pathogen includes SARS-CoV-2, and the detectable target antigen includes the N protein and/or the S protein.

According to some aspects, the positive control of the present disclosure may be provided with at least two quantification values. According to some aspects, the at least two quantification values may be selected from the TCID₅₀ value of the pathogen in the culture fluid prior to inactivation, the molecular concentration of the pathogen in the culture fluid, the first concentration of detectable target antigen, and the second concentration of detectable target antigen. For example, the positive control may be provided in or with a container having the at least two quantification values provided thereon or therewith. The present disclosure is also directed to a kit containing the positive control and instructions having the at least two quantification values as described herein.

According to some aspects, the positive control may be storable at nonfrozen temperatures, such as from 2-8° C. According to some aspects, the positive control may be storable at room temperature, such as from 20-22° C. According to some aspects, the positive control may be storable at frozen temperatures, such as below −65° C.

The present disclosure is also directed to a method of using the positive control as described herein in an antigen biomarker assay.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Herein, the recitation of numerical ranges by endpoints (e.g. between about 50:1 and 1:1, between about 100 and 500° C., between about 1 minute and 60 minutes) include all numbers subsumed within that range, for example, between about 1 minute and 60 minutes includes 21, 22, 23, and 24 minutes as endpoints within the specified range. Thus, for example, ranges 22-36, 25-32, 23-29, etc. are also ranges with endpoints subsumed within the range 1-60 depending on the starting materials used, temperature, specific applications, specific embodiments, or limitations of the claims if needed. The Examples and methods disclosed herein demonstrate the recited ranges subsume every point within the ranges because different synthetic products result from changing one or more reaction parameters. Further, the Examples and methods disclosed herein describe various aspects of the disclosed ranges and the effects if the ranges are changed individually or in combination with other recited ranges.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

As used herein, the terms “about” and “approximately” are defined to being close to as understood by one of ordinary skill in the art. In one non-limiting embodiment, the terms “about” and “approximately” are defined to be within 10%, preferably within 5%, more preferably within 1%, and most preferably within 0.5%.

The examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### EXAMPLES

### Example I: Preparation of Positive Control Using SARS-CoV-2

First, a stock of frozen cell culture was thawed to provide a culture fluid containing SARS-CoV-2, and the N protein concentration of the culture fluid was determined via ELISA. The frozen cell culture included the target viral pathogen (i.e., SARS-CoV-2), the host cells used to propagate the viral pathogen, and the culture media used for growth of the viral pathogen. To thaw the frozen cell culture, tubes containing the frozen cell culture were removed from a freezer (in this example, an ultracold freezer at a temperature of −65° C. or below) and thawed in a water bath for a time of 30 minutes or less. The culture fluid was then clarified of cell debris by centrifugation in a centrifuge for 10 minutes at 1500 rpm or 500×g. The supernatant was collected and pooled. Then, aliquots of the culture fluid were placed in sterile petri dishes. The petri dishes were each 245 mm mm×26 mm and contained 25-45 mL of clarified culture fluid, although other petri dish sizes and sample volumes are acceptable. Table 1 below shows the maximum volume capacity of various petri dishes by size.

TABLE 1
Maximum Volume Capacity of Petri Dishes by Size.

| Petri Dish Size | Max Capacity | |
|---|---|---|
| 100 mm × 15 mm | 5 | mL |
| 150 mm × 15 mm | 7.5 | mL |
| 245 mm × 26 mm | 45 | mL |

Each aliquot was then exposed to UV irradiation using a 45 Model G30T8 Ultraviolet UV-C Lamp at a wavelength of 254 nm for about 5 minutes. Each aliquot was stirred and then again exposed to UV irradiation using the 45 Model G30T8 Ultraviolet UV-C Lamp at a wavelength of 254 nm for about 5 minutes to provide aliquots of the positive control. The aliquots were then pooled and inactivation of the pathogen was determined using an Infectivity assay. The N protein concentration of the positive control was determined via ELISA.

### Example II: Preparation of Positive Control Using Influenza a H1N1Pdm Virus

Three independent lots of a culture fluid containing Influenza A H1N1pdm Virus (Strain Gaungdong-Maonan-SWL 1536/19) were divided into aliquots. Each aliquot was then subjected to inactivation conditions as shown in Table 2. One aliquot was not subjected to inactivation conditions in order to serve as a control.

TABLE 2
Inactivation Conditions

| Sample | Inactivation Condition |
|---|---|
| CF | None |
| CFHI | Heat (56° C. for 1 hour) |
| UV-5 minutes | Exposure to UV light for 5 minutes |
| UV-10 minutes | Exposure to UV light for 10 minutes |
| UV-15 minutes | Exposure to UV light for 15 minutes |

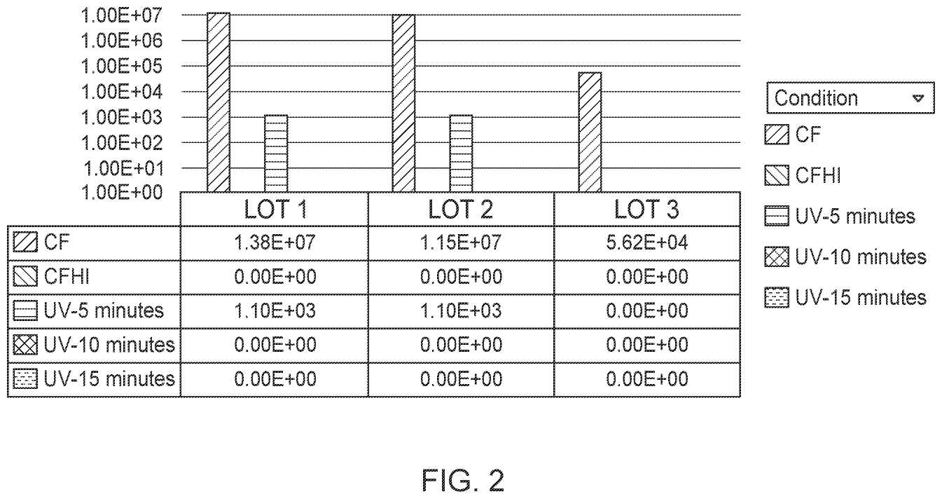

TCID₅₀ measurements were then conducted for each sample. The results of these measurements are shown in FIG. 2. It was determined that UV exposure for a time of 10 or 15 minutes was sufficient to inactivate the pathogen, as demonstrated by the absence of any recoverable TCID₅₀ units.

### Example III: Preparation of Positive Control Using SARS-COV2 Virus

Three independent lots of a culture fluid containing SARS-COV2 Virus (USA-WA 1/2020 strain) were divided into aliquots. Then, each aliquot was subjected to inactivation conditions as shown in Table 3. One aliquot was not subjected to inactivation conditions in order to serve as a control.

TABLE 3
Inactivation Conditions

| Sample | Inactivation Condition |
|---|---|
| CF | None |
| CFHI | Heat (60° C. for 1 hour) |
| UV-5 minutes | Exposure to UV light for 5 minutes |
| UV-10 minutes | Exposure to UV light for 10 minutes |
| UV-15 minutes | Exposure to UV light for 15 minutes |

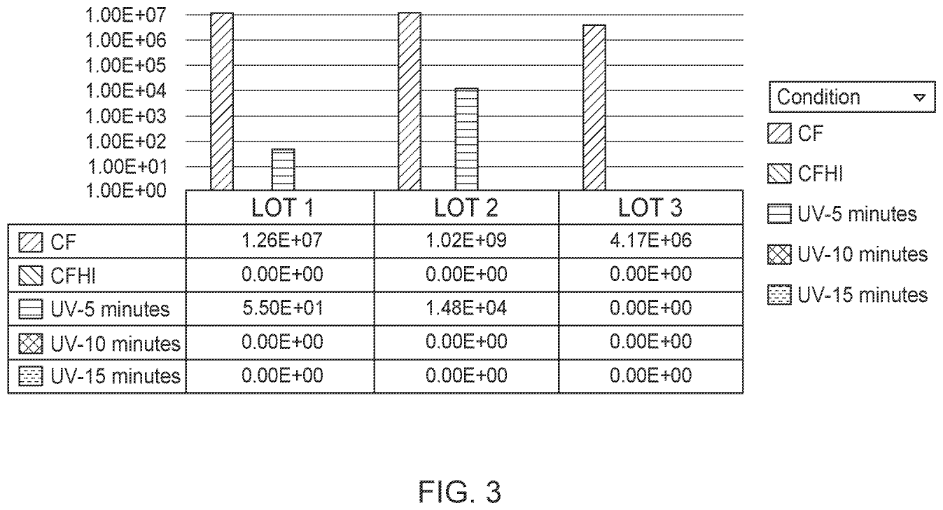

TCID₅₀ measurements were then conducted for each sample. The results of these measurements are shown in FIG. 3. It was determined that UV exposure for a time of 10 or 15 minutes was sufficient to inactivate the pathogen, as demonstrated by the absence of any recoverable TCID₅₀ units.

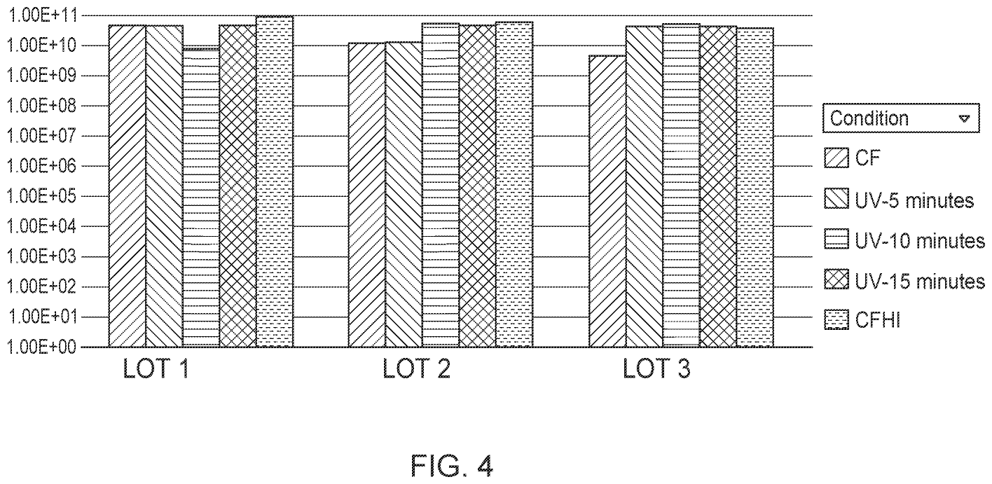

SARS-CoV-2 qPCR was then performed in order to determine the impact of the inactivation conditions on nucleic acid integrity. FIG. 4 shows the results of this analysis. As shown, the qPCR signals across all samples were consistent. It was therefore concluded that UV inactivation results in no adverse effects on viral nucleic acid targeted in this test.

Finally, the N protein concentration of each sample after inactivation was determined via ELISA. To determine the N protein concentration, first, a standard curve was prepared utilizing 200 ng/mL of SARS-CoV-2 NP standard as shown in Table 4.

TABLE 4
Preparation of SARS-CoV-2 NP Antigen Standard

| | Concentration of | SARS-CoV-2 NP | Assay |
|---|---|---|---|
| Standard | SARS-CoV-2 NP | Antigen Standard | Diluent |
| Number | (ng/mL) | (μL) | (μL) |
| 1 | 8.0 | 40 | 960 |
| 2 | 4.0 | 500 of #1 | 500 |
| 3 | 2.0 | 500 of #2 | 500 |
| 4 | 1.0 | 500 of #3 | 500 |
| 5 | 0.5 | 500 of #4 | 500 |
| 6 | 0.25 | 500 of #5 | 500 |
| 7 | 0 | 0 | 500 |

Then, each sample was diluted in triplicate with an assay diluent. A SARS-CoV-2 anti NP microplate was then prewashed three times using 350 μL of a plate wash buffer, aspirating between washes. The microplate was then struck on an absorbent towel until no droplets remained in the wells, although the wells were not allowed to completely dry. Then, 200 μL of either the SARS-CoV-2 NP Standard or a sample was added to each well, and the microplate was incubated at 37° C. for 1.5 hours.

The wells were then aspirated and washed six times using 350 μL of the plate wash buffer, aspirating between washes. The microplate was then struck on an absorbent towel until no droplets remained in the wells, although the wells were not allowed to completely dry.

Then, 100 μL of an SARS-CoV-2 NP detector antibody was added to each well, and the microplate was incubated at 37° C. for 1 hour. The wells were then aspirated and washed six times using 350 μL of the plate wash buffer, aspirating between washes. The microplate was then struck on an absorbent towel until no droplets remained in the wells, although the wells were not allowed to completely dry.

Then, 100 μL of a high-sensitive TMB substrate was added to each well, and the microplate was incubated at room temperature (20-25° C.) for 30 minutes. 100 μL of a stop solution was then added to each well, and the microplates were read at 450 nm on a plate reader.

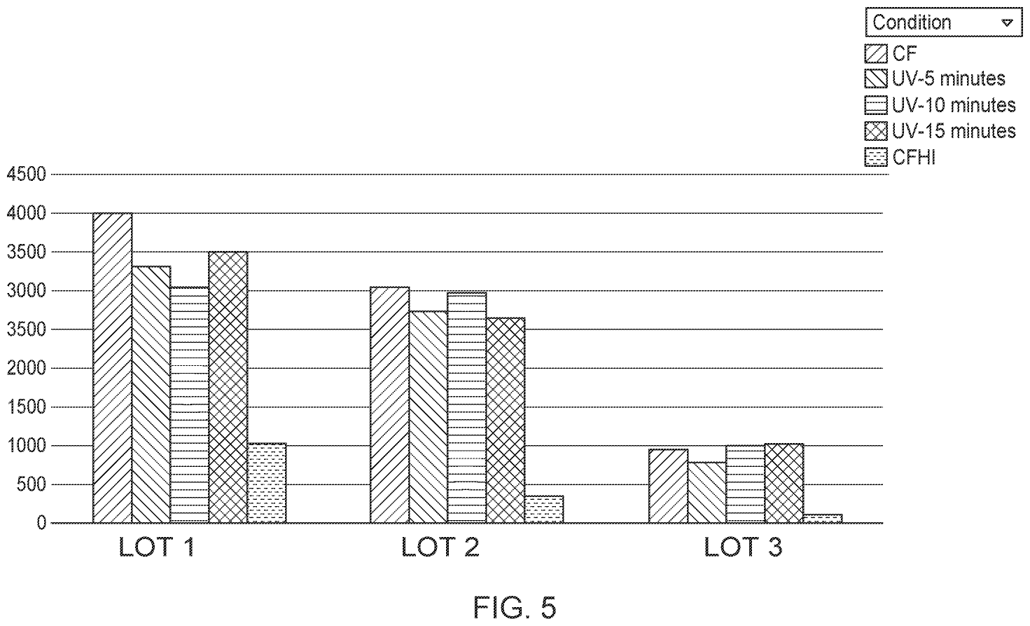

The results of this analysis are shown in FIG. 5. As shown in FIG. 5, the N protein concentration after heat inactivation is significantly lower than the N protein concentration of the control (i.e., the sample that was not inactivated). However, FIG. 5 shows that UV inactivation provided a minimal change in N protein concentration as compared with the control.

What is claimed is:

1. A method for producing a positive antigen-based control for detecting a pathogen, comprising:
providing a culture fluid comprising a pathogen having a first detectable antigen, wherein the culture fluid has a first concentration of the first detectable antigen; and
exposing the culture fluid to electromagnetic irradiation for a time period sufficient to render the pathogen inactivated, thereby producing a positive antigen-based control,
wherein the positive antigen-based control comprises a second concentration of the first detectable antigen that is no more than about 50% different from the first concentration when determined via ELISA.

2. The method of claim 1, wherein the pathogen comprises a virus.

3. The method of claim 2, wherein the virus comprises SARS-CoV-2, RSV, Flu A, Flu B, or a combination thereof.

4. The method of claim 3, wherein the detectable target antigen comprises a nucleocapsid protein.

5. The method of claim 1, wherein the electromagnetic irradiation comprises UV irradiation provided by UV-C light.

6. The method of claim 1, further comprising combining the culture fluid with a protease inhibitor.

7. The method of claim 6, wherein the positive antigen-based control comprises the protease inhibitor.

8. The method of claim 1, further comprising combining the positive antigen-based control with a protease inhibitor.

9. The method of claim 1, wherein the time period is between about 1 and 15 minutes.

10. A kit comprising:
a positive antigen-based control, the positive antigen-based control comprising an inactivated pathogen; and
at least two quantification values.

11. The kit of claim 10, wherein the pathogen comprises a virus.

12. The kit of claim 11, wherein the virus comprises SARS-CoV-2, RSV, Flu A, Flu B, or a combination thereof.

13. The kit of claim 10, wherein the at least two quantification values comprise a TCID₅₀ value of the pathogen prior to inactivation and a concentration of a detectable target antigen.

14. The kit of claim 10, wherein the virus comprises SARS-CoV-2, and wherein the detectable target antigen is a nucleocapsid protein.

15. The kit of claim 11, further comprising a protease inhibitor.

16. The kit of claim 11, wherein the inactivated pathogen is provided in a fluid.

17. A positive antigen-based control for detecting a pathogen, wherein the positive antigen-based control is provided by a method comprising:
providing a culture fluid comprising a pathogen, wherein the culture fluid has a detectable target antigen at a first concentration; and
exposing the culture fluid to electromagnetic irradiation for a time period sufficient to render the pathogen inactivated, thereby producing the positive antigen-based control,
wherein the positive antigen-based control comprises a second concentration of the detectable target antigen that is no more than about 50% different from the first concentration, and
wherein the first concentration and the second concentration are determinable via ELISA.

18. The positive antigen-based control of claim 17, wherein the pathogen comprises a virus.

19. The positive antigen-based control of claim 18, wherein the virus comprises SARS-CoV-2, RSV, Flu A, Flu B, or a combination thereof.

20. The positive antigen-based control of claim 19, wherein the detectable target antigen comprises a nucleocapsid protein.
